(19) United States
(12) Patent Application Publication
(10) Pub. No.: US 2024/0173699 A1
(43) Pub. Date: May 30, 2024

(54) PROCESSES FOR ACTIVATING DEHYDROGENATION CATALYST

(71) Applicant: UOP LLC, Des Plaines, IL (US)

(72) Inventors: Xi Zhao, Arlington Heights, IL (US); Ling Zhou, Palatine, IL (US); Wei Pan, Hoffman Estates, IL (US)

(21) Appl. No.: 18/457,059

(22) Filed: Aug. 28, 2023

Related U.S. Application Data
(60) Provisional application No. 63/385,497, filed on Nov. 30, 2022.

(51) Int. Cl. B01J 23/62; B01J 37/08; B01J 38/02

(52) U.S. Cl. CPC B01J 23/62; B01J 37/08; B01J 38/02

(57) ABSTRACT

Processes and apparatuses for activating a dehydrogenation catalyst that include gallium on a support. An additive particle comprising platinum is mixed with the catalyst particles, which may be fresh or deactivated catalyst. The mixture is exposed, in an oxygen containing environment, to a temperature between 650° C. and 1,000° C. The additive particles may be fluidized with the catalyst particles and may active the catalyst in a regenerator.

### RELATED APPLICATIONS

This application claims priority to U.S. Provisional Patent Application Ser. No. 63/385,497 filed on Nov. 30, 2022, the entirety of which is incorporated herein by reference.

### FIELD OF THE INVENTION

This invention relates generally to processes for activating dehydrogenation catalysts, and more particularly to processes which use a platinum containing particle, different from the catalyst, to activate the catalyst.

### BACKGROUND OF THE INVENTION

Catalytic dehydrogenation reactions are important industrial processes which generate olefins including propylene, butene, styrene, to name a few.

The reaction of light paraffin dehydrogenation is often thermodynamically limited and has lower one-pass conversion at lower temperature. To increase one-pass conversion and overall process efficiency, dehydrogenation catalyst having both high activity and compatibility with high reaction temperature is needed to allow dehydrogenation process operation under more favorable equilibrium. Furthermore, it is also desirable for the catalyst to be able to tolerate even higher regeneration temperature and rapid cycling so that it can also provide reaction heat and limit thermal residence time of feed stream.

One example of a suitable material is a supported gallium catalyst. It is known that adding platinum to the supported gallium catalyst increases the dehydrogenation activity. Platinum can be added to the gallium catalyst, for example, with the process described in U.S. Pat. No. 9,834,496.

However, the severe dehydrogenation process conditions often result in activity loss for the catalyst. There is a general desire to activate and/or reactivate dehydrogenation catalyst and maintain its activity during the physical life of catalyst particles.

U.S. Patent Application No. 2021/0129117A1 discloses a process of reconstituting a deactivated Pt—Ga catalyst by impregnation of a solution containing Pt salt on to the deactivated catalyst, followed by calcination. However, such a reconstitution procedure requires the catalyst to be removed from the operation and cooled to allow the solution impregnation step. Thus, the procedure causes interruption to the continuous operation of dehydrogenation process.

Accordingly, it would be desirable to have more effective and efficient ways to activate or re-activate dehydrogenation catalyst.

### SUMMARY OF THE INVENTION

The present inventors have discovered a process to activate or re-activate a Ga or Pt—Ga based dehydrogenation catalyst (“catalyst”). The process comprises of mixing the Ga or Pt—Ga catalyst with solid particles containing Pt (“additive”), and subsequent calcination in an oxygen containing environment. The additive can be removed from mixture or remain in the mixture after the calcination treatment, and the mixture after treatment will exhibit increased dehydrogenation activity.

Therefore, the present invention may be characterized, in at least one aspect, as providing a process for activating a dehydrogenation catalyst by: providing dehydrogenation catalyst particles comprising gallium, and optionally platinum, on a support; providing an additive particle comprising platinum, wherein a level of platinum on the additive particle is at least 0.005 wt %, and when the dehydrogenation catalyst particles comprise platinum, wherein the level of platinum on the additive particle is higher than the platinum level on the dehydrogenation catalyst particles; treating the catalyst particles and the additive particle simultaneously in an oxygen containing gas environment at a temperature between 650° C. and 1,000° C.; and, wherein an improvement of dehydrogenation activity is observed in: the dehydrogenation activity of the catalyst particles increases after the treatment and is higher than that of the catalyst particles before the treatment in otherwise identical dehydrogenation reaction conditions; or, the dehydrogenation activity of the mixture increases after the treating and is higher than that of the mixture before the treating in otherwise identical dehydrogenation reaction conditions, or both.

The treating temperature may be between 700° C. and 900° C.

The dehydrogenation catalyst particles may be fresh catalyst when introduced to the additive particle.

The dehydrogenation catalyst particles may be deactivated catalyst.

The oxygen concentration in the gas environment during treating may be at least 0.1 mol %.

The treating may be performed in a catalyst regenerator.

The treating may be performed simultaneously as regenerating and re-heating the dehydrogenation catalyst particles.

The process may also include separating the dehydrogenation catalyst particles from the additive particle after the treating.

The support for the dehydrogenation catalyst particles may be or include silica, alumina, silica-alumina, or amorphous aluminum phosphate.

The additive particle may further include a support, and the support for the additive particle may be or include silica, alumina, silica-alumina, titania, zirconia, ceria, or amorphous aluminum phosphate.

The mixture may be fluidized during treating.

In another aspects, the present invention may be generally characterized as providing a process for activating a dehydrogenation catalyst by: providing dehydrogenation catalyst particles in a regeneration zone, wherein the catalyst particles comprise gallium, and optionally platinum, on a support, and wherein the regeneration zone is configured to receive an oxygen containing stream and an optional fuel stream and operates under conditions to re-heat the catalyst particles; providing additive particle comprising platinum on a support, wherein a level of platinum on each additive particle is at least 0.005 wt %, and when the dehydrogenation catalyst particles comprise platinum, wherein the level of platinum on the additive particle is higher than the platinum level on the dehydrogenation catalyst particles; treating the catalyst particles and additive particle simultaneously in the regeneration zone in an oxygen containing environment at a temperature between 650° C. and 1,000° C.; wherein an improvement of dehydrogenation activity is observed in: the dehydrogenation activity of the catalyst particles increases after the treatment and is higher than that of the catalyst particles before the treatment in otherwise identical dehydrogenation reaction conditions, or the dehydrogenation activity of the mixture increases after the treating and is higher than that of the mixture before the treating in otherwise identical dehydrogenation reaction conditions, or both.

The treating may be performed simultaneously as regenerating and re-heating the catalyst particles.

The temperature may be between 700° C. and 900° C.

The dehydrogenation catalyst particles may be fresh catalyst when mixed with the additive particles.

The dehydrogenation catalyst particles may be deactivated catalyst.

An oxygen concentration in the regenerator zone during treatment may be at least 0.1 mol %.

In a third aspect, the present invention may be broadly characterized as providing a mixture of: dehydrogenation catalyst particles comprising gallium, and optionally platinum, on a support; and, additive particles comprising platinum on a support, wherein a level of platinum on each additive particle is at least 0.005 wt %; and when the dehydrogenation catalyst particles comprise platinum, wherein the level of platinum on the additive particle is higher than the platinum level on the dehydrogenation catalyst particles. The mixture or the dehydrogenation catalyst particles or both have an increased dehydrogenation activity after exposed to an oxygen containing environment at a temperature between 650° C. and 1,000° C., when tested in otherwise identical dehydrogenation reaction conditions.

The support for the dehydrogenation catalyst particles and the support for the additive particles each, independently, may be silica, alumina, silica-alumina, titania, zirconia, ceria, or amorphous aluminum phosphate.

The mixture may be fluidizable.

Additional aspects, embodiments, and details of the invention, all of which may be combinable in any manner, are set forth in the following detailed description of the invention.

### DETAILED DESCRIPTION OF THE DRAWINGS

One or more exemplary embodiments of the present invention will be described below in conjunction with the following drawing figures, in which:

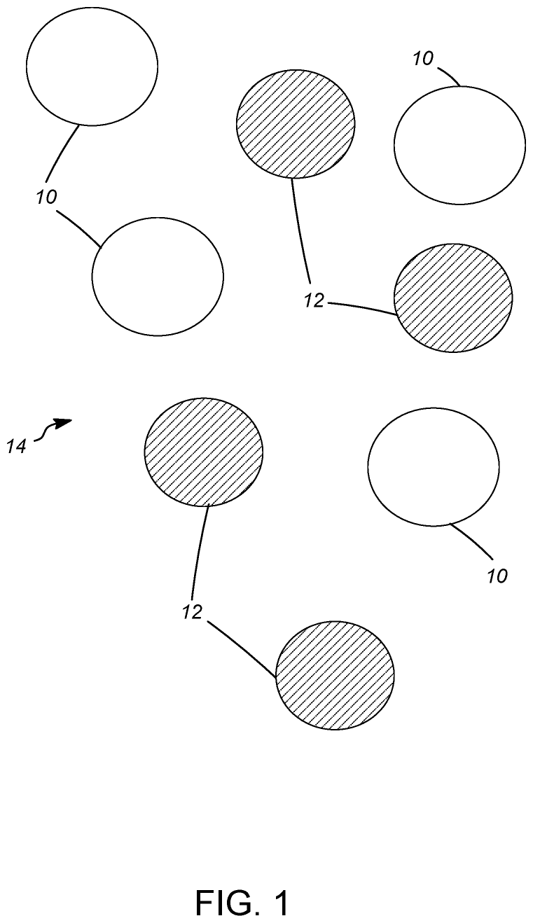

FIG. 1 is a schematic drawing of a mixture of catalyst and additive particles according to one or more aspects of the present invention.

### DETAILED DESCRIPTION OF THE INVENTION

As mentioned above, processes to activate, either initially or as reactivation, dehydrogenation catalysts with Pt containing particles and a calcination in an oxygen containing environment have been invented. The activation may be done in situ-without requiring removal of the catalyst or shutdown of operations. Additionally, since the continuous operation of dehydrogenation process generally involves a step during which the catalyst is exposed to oxygen containing environment for purposes such as coke-burning, re-oxidation or re-heating, the activation may be accomplished during this step of dehydrogenation processing, without requiring additional operation steps in the process cycle.

With these general principles in mind, one or more embodiments of the present invention will be described with the understanding that the following description is not intended to be limiting.

Dehydrogenation of hydrocarbon can be carried out in different types of reactors (for example, fixed bed, moving bed or circulating fluidized bed) and with different catalyst. The present invention may be used with any of these reactor types but is believed to be particularly beneficial in circulating fluidized bed processes.

Dehydrogenation catalysts with supported Pt—Ga are known to exhibit dehydrogenation activity across a wide range of Pt/Ga atomic ratios. The composition typically has relatively low Pt level (typically between 1 and 1500 ppmw) compared to Ga level (typically 0.25 wt % to 5 wt %).

The support used for the catalyst typically comprises silica, alumina, silica-alumina, or amorphous aluminum phosphate.

During commercial operation of a dehydrogenation process, for example in a circulating fluidized bed, the supported Pt—Ga catalyst typically cycles between approximately 600° C. and approximately 750° C. It is known that over time the catalyst loses Pt and/or loses activity. Therefore, if an aqueous solution impregnation method is used to add Pt to the working catalyst that experienced reduced activity, the catalyst first needs to be removed from operation cycle and cooled before the impregnation to prevent rapid vaporization of liquid upon contact with solution. This causes interruption to commercial operation.

To overcome this disruption, as shown in FIG. 1, the present inventors have discovered processes in which, instead of using liquid Pt solution impregnation, catalyst particles 10 are activated by mixing, blending, or otherwise contacting the catalyst particles 10 with solid additive particles 12 containing Pt, preferably at a higher Pt level than the catalyst particles 10, to form a mixture 14. After mixing, blending, or otherwise contacting the two particles 10, 12 together, the mixture 14 is subjected to a heat treatment, such as calcination, in an oxygen containing environment. Typically, the oxygen containing environment has at least 0.1% oxygen in the gas phase, for example from 0.5% to 100% O₂.

The heat treatment procedure is generally carried out between 650° C. and 1,000° C. in oxidative environment, or more preferably, between 700° C. and 900° C. The mixture during heat treatment could be stationary (such as in an oven or fixed bed) or moving (such as in a kiln or in a fluidized bed), with or without environmental gas stream actively passing over or through it.

The additive can be of substantially different size and shape to the catalyst, and if necessary, removed after the heat treatment, or the additive can have similar size to the catalyst and not removed. If the additive is not removed, it should not introduce undesired reaction under dehydrogenation condition (i.e., has negligible catalytic cracking activity).

For example, in a fluidized bed dehydrogenation process, a small amount of fluidizable additive loaded with Pt may be introduced into the reactor or regenerator. The introduction of the additive can be carried out using typical apparatus and procedures of adding make-up dehydrogenation catalyst, either alone or pre-mixed with the make-up dehydrogenation catalyst. The additive is preferably attrition resistant and will circulate together with the working catalyst. It will not interfere with the dehydrogenation reactions and will activate the working catalyst in the regeneration or other non-dehydrogenation steps at high temperature in the oxygen containing environment.

It is further contemplated that the Pt additive is contained in baskets or other porous containers and installed in fixed locations within the regenerator or other process locations exposing catalyst to oxygen containing environment. This would allow for the utilization of additives with larger size than fluidized catalyst at designated locations with high temperature and oxygen gas environment. While the larger sized additive remains stationary, working dehydrogenation catalyst could pass through the region and be in proximity or in direct contact with the additive.

The activation may be performed on new or fresh catalyst or on catalyst that initially does not have Pt. The activation may also be performed on deactivated catalyst, or catalyst that has a reduced amount of Pt compared with initial levels and/or has lost activity after one or more cycles of operation and is no longer reach initial high activity even after coke-burning/regeneration. Accordingly, as used herein, “activation” or “activating” should be understood to also include “reactivation” or “reactivating.”

The additive particle may or may not include gallium. The additive particle may include a porous support, such as a silica, alumina, silica-alumina, or amorphous aluminum phosphate support.

To effectively activate the catalyst, the concentration of platinum on the additive particles should be higher that the concentration of platinum on the catalyst particles, or no less than 50 ppm by weight, whichever is greater. Generally, a level of platinum on the additive particle is at least 0.005 wt %.

### EXAMPLES

### Example 1: Pt Loss of Pt—Ga Catalyst after Exposure to High Temperature Oxidative Environment

A solution of Tetraammineplatinum chloride (TAPC), Ga nitrate and KNO₃ in water was impregnated onto a calcined spray dried silica-alumina support by incipient wetness method, followed by 705° C. 4-hour calcination in air to remove water and decompose the salt. The resulting catalyst A has the metal loading listed in TABLE 1 below. The catalyst A is then exposed to 900° C. for 1 hour in air, and the composition of impregnated metals are measured again after the 900° C. exposure and listed in TABLE 1. As shown in TABLE 1 below, significant Pt loss is observed while the level of Ga and K remains virtually unchanged.

TABLE 1

| | Catalyst A | Catalyst A |
|---|---|---|
| | before 900° C. 1 h | after 900° C. 1 h |
| | calcination | calcination |
| Pt/ppm wt | 87 | 30 |
| Ga/wt % | 1.36 | 1.38 |
| K/wt % | 0.25 | 0.25 |

### Example 2: Pt Loss after Propane Dehydrogenation Reaction-Regeneration Cycles

A solution of TAPC, Ga nitrate, and KNO₃ in water was impregnated onto a calcined spray dried Silica-alumina support by incipient wetness method, followed by 705° C. 4-hour calcination in air to remove water and decompose the salt. The calcined catalyst B was then cycled between dehydrogenation and regeneration at the following conditions in a fixed-bed reactor system:

0.85 g of catalyst was loaded in a quartz reactor with 8 mm ID. the catalyst was heated to 720° C. in nitrogen atmosphere, and then went through cycles of dehydrogenation and regeneration. Each reaction-regeneration cycle consists of:

1. A regeneration step of 2 min in a gas mixture with a composition of 24.2 mol % steam, 2.4 mol % oxygen, and balance nitrogen at 720° C. The gas flow was about 30 cc/min.

2. Cooling in a nitrogen atmosphere at 13° C./min to 620° C.

3. A dehydrogenation reaction step of 2 min at 620° C., under ambient pressure with a propane feed flow rate of about 15 cc/min.

4. Heating in a nitrogen atmosphere at 10° C./min to 720° C., and repeating steps 1-4.

After 1,575 cycles, the spent catalyst was recovered, and the remaining metal level measured and compared with the starting catalyst in Table 2. The results showed that like the high temperature calcination experiment in Example 1, cycle-operation of dehydrogenation-regeneration at up to 720 C also resulted in loss of Pt but no loss of Ga or K for a typical Pt—Ga dehydrogenation catalyst.

TABLE 2

| | | Catalyst B after 1575 cycles of |
|---|---|---|
| | Catalyst B | operation |
| Pt/ppm wt | 76 | 48 |
| Ga/wt % | 1.56 | 1.51 |
| K/wt % | 0.26 | 0.26 |

### Example 3: Activation of Supported Ga Catalyst by Fluidized Pt Additive

A solution of Ga nitrate and KOH was added to a slurry of silica-alumina and spray dried. The spray dried product was calcined at 704° C. in air with 2% moisture for 10 hour and then 899° C. in dry air for 1 hour. The product was found to contain 1.2 wt % Ga, 0.25 wt % K and 1 wt % SiO₂. This catalyst was designated as catalyst C.

A solution of TAPC and KNO₃ was added to a spray dried alumina using incipient wetness method, and the spray dried alumina was subsequently calcined at 500° ° C. for 30 min. The product was determined to contain 0.01 wt % Pt and 0.2 wt % K, and is designated as additive A.

Catalyst C and additive A were mixed thoroughly at a mass ratio of 9:1, then calcined in a crucible at 750° C. for 3 hours in air to obtain Activated Catalyst D. Another mixture of catalyst C and additive A at the same ratio was calcined in a crucible at 900° ° C. for 21.25 hours in air to obtain Activated Catalyst E.

### Example 4: Dehydrogenation Activity Testing

The following procedure was used to evaluate the catalyst activity for propane dehydrogenation reaction:

A sample of 200 mg catalyst was loaded into a quartz reactor (ID=7 mm) between two layers of quartz wool support. The sample was pretreated at 635° C. for 60 min under nitrogen and cooled to 620° C. under nitrogen flow. Dehydrogenation reaction was carried out with 20 standard cubic centimeter per minute (SCCM) of pure propane flow at 620° C. under atmospheric pressure. The product was analyzed by gas chromatograph equipped with GS-GasPro and HP-PONA columns and FID detector for methane, ethane, ethylene, propane, propylene, and C4-plus hydrocarbon species at 3.5 minutes after feed introduction.

The performance of catalyst C, additive A, their 9:1 physical mixture without any heat treatment, activated catalyst D and activated catalyst E are listed in TABLE 3 below:

TABLE 3

| | | | 9:1 physical | | Activated |
|---|---|---|---|---|---|
| | | | mixture of | Activated | catalyst E |
| | | Addi- | Catalyst C | catalyst D | (900 C. |
| | Catalyst | tive | & Additive | (750 C. 3 | 21.25 |
| Cat Name | C | A | A | hours) | hours) |
| Propane | 15.58 | 12.98 | 14.05 | 38.87 | 43.15 |
| Conversion | | | | | |
| @ 3.5 MOS | | | | | |
| Propylene | 87.68 | 86.25 | 86.39 | 97.40 | 97.98 |
| Selectivity | | | | | |
| @ 3.5 MOS | | | | | |

It can be observed that catalyst C without any Pt showed a low activity. Additive A on its own, or the physical mixture of catalyst C and additive A, is less active than catalyst C. The treatment at 750° C. for 3 hours resulted in significant activation of catalyst/additive mixture, which now shows higher propane conversion and higher propylene selectivity than the starting catalyst C. Treatment for higher temperature and longer period resulted in further incremental benefit on both activity and selectivity.

### Example 5: Activation of Supported Ga Catalyst by Large Pill Pt Additive

Catalyst F was made following the procedure of catalyst C in example 3, except that the level of Ga nitrate was adjusted to obtain 0.79 wt % Ga in final catalyst.

Additive B was made by incipient wetness impregnation of TAPC onto spherical gamma-alumina particles of 2 mm diameter, followed by calcination at 500° C. for 3 hours. The calcined spherical alumina particles have 0.5 wt % Pt.

Catalyst F and additive B was mixed at a mass ratio of 22.5:1 and loaded in a quartz fluidized bed (ID=54 mm). The mixture was fluidized in air at 1,200 gas-hourly space velocity (GHSV) for 8 hours at 750° C. under atmospheric pressure, then cooled down and passed through sieve of proper size to remove all additive B particles. The recovered fluidized catalyst was named catalyst G. Another mixture of catalyst F and additive B at the same mass ratio was treated in the same fluidized bed under the same condition, except of temperature at 800° C., and sieved to recover the fluidizable catalyst as activated catalyst H.

Catalyst F and additive A (from example 3) was mixed at a mass ratio of 9:1 and treated in the same way as activated catalyst G (in quartz fluidized bed at 750° C. and air GHSV=1,200 for 8 hours), but not sieved afterwards to separate the additive particles from catalyst particles. The treated mixture was called activated catalyst I.

The above activated catalysts are tested following the same procedure in example 4 and the results are compared in table 4 below:

TABLE 4

| | Catalyst | Activated | Activated | Activated |
|---|---|---|---|---|
| Cat Name | F | Catalyst G | Catalyst H | Catalyst I |
| Activation | N/A | Additive B | Additive B | Additive A |
| condition | | (removed after | (removed after | 750 C. 8 h |
| | | activation) | activation) | |
| | | 750 C. 8 h | 800 C. 8 h | |
| Propane | 17.76 | 29.06 | 36.23 | 41.52 |
| Conversion @ | | | | |
| 3.5 MOS | | | | |
| Propylene | 89.57 | 96.47 | 97.68 | 97.78 |
| Selectivity | | | | |
| @ 3.5 MOS | | | | |

It can be seen from the results that catalyst F itself has similar low dehydrogenation activity without activation. When mixed with Pt supported on large alumina sphere and treated in fluidized bed, catalyst F could gain significant activity despite the larger size of additive B and less favorable solid-solid contact in fluidized bed condition. It can also be further seen that the activity improvement is due to the activation of catalyst F and not additive B itself since the additive particles are physically removed from activated catalyst G and H before testing. When comparing the activation between activated catalysts G, H, and I as well as D and E, one could conclude that the degree of activation is related to both physical status of additive (including but not limited to particle size and Pt level) and activation conditions (including but not limited to temperature, catalyst motion, oxygen level, gas velocity, pressure, and time).

### Specific Embodiments

While the following is described in conjunction with specific embodiments, it will be understood that this description is intended to illustrate and not limit the scope of the preceding description and the appended claims.

A first embodiment of the invention is a process for activating a dehydrogenation catalyst, the process comprising providing dehydrogenation catalyst particles comprising gallium, and optionally platinum, on a support, providing an additive particle comprising platinum, wherein a level of platinum on the additive particle is at least 0.005 wt %, and when the dehydrogenation catalyst particles comprise platinum, wherein the level of platinum on the additive particle is higher than the platinum level on the dehydrogenation catalyst particles, treating the catalyst particles and the additive particle simultaneously in an oxygen containing gas environment at a temperature between 650° ° C. and 1,000° C.; wherein an improvement of dehydrogenation activity is observed in at least one of the dehydrogenation activity of the catalyst particles increases after the treatment and is higher than that of the catalyst particles before the treatment in otherwise identical dehydrogenation reaction conditions, or the dehydrogenation activity of the mixture increases after the treating and is higher than that of the mixture before the treating in otherwise identical dehydrogenation reaction conditions. An embodiment of the invention is one, any or all of prior embodiments in this paragraph up through the first embodiment in this paragraph, wherein the treating temperature is between 700° C. and 900° C. An embodiment of the invention is one, any or all of prior embodiments in this paragraph up through the first embodiment in this paragraph, wherein the dehydrogenation catalyst particles are fresh catalyst when introduced to the additive particle. An embodiment of the invention is one, any or all of prior embodiments in this paragraph up through the first embodiment in this paragraph, wherein the dehydrogenation catalyst particles are deactivated catalyst. An embodiment of the invention is one, any or all of prior embodiments in this paragraph up through the first embodiment in this paragraph, wherein the oxygen concentration in the gas environment during treating is at least 0.1 mol %. An embodiment of the invention is one, any or all of prior embodiments in this paragraph up through the first embodiment in this paragraph, wherein the treating is performed in a catalyst regenerator. An embodiment of the invention is one, any or all of prior embodiments in this paragraph up through the first embodiment in this paragraph, wherein the treating is performed simultaneously as regenerating and re-heating the dehydrogenation catalyst particles. An embodiment of the invention is one, any or all of prior embodiments in this paragraph up through the first embodiment in this paragraph further comprising separating the dehydrogenation catalyst particles from the additive particle after the treating. An embodiment of the invention is one, any or all of prior embodiments in this paragraph up through the first embodiment in this paragraph wherein the support for the dehydrogenation catalyst particles comprises silica, alumina, silica-alumina, or amorphous aluminum phosphate. An embodiment of the invention is one, any or all of prior embodiments in this paragraph up through the first embodiment in this paragraph wherein the additive particle further comprises a support, and the support for the additive particle comprises silica, alumina, silica-alumina, titania, zirconia, ceria, or amorphous aluminum phosphate. An embodiment of the invention is one, any or all of prior embodiments in this paragraph up through the first embodiment in this paragraph wherein the mixture is fluidized during treating.

A second embodiment of the invention is a process for activating a dehydrogenation catalyst, the process comprising providing dehydrogenation catalyst particles in a regeneration zone, wherein the catalyst particles comprise gallium, and optionally platinum, on a support, and wherein the regeneration zone is configured to receive an oxygen containing stream and an optional fuel stream and operates under conditions to re-heat the catalyst particles; providing additive particle comprising platinum on a support, wherein a level of platinum on each additive particle is at least 0.005 wt %, and when the dehydrogenation catalyst particles comprise platinum, wherein the level of platinum on the additive particle is higher than the platinum level on the dehydrogenation catalyst particles, treating the catalyst particles and additive particle simultaneously in the regeneration zone in an oxygen containing environment at a temperature between 650° C. and 1,000° C.; wherein an improvement of dehydrogenation activity is observed in at least one of the dehydrogenation activity of the catalyst particles increases after the treatment and is higher than that of the catalyst particles before the treatment in otherwise identical dehydrogenation reaction conditions, or the dehydrogenation activity of the mixture increases after the treating and is higher than that of the mixture before the treating in otherwise identical dehydrogenation reaction conditions. An embodiment of the invention is one, any or all of prior embodiments in this paragraph up through the second embodiment in this paragraph, wherein the treating is performed simultaneously as regenerating and re-heating the catalyst particles. An embodiment of the invention is one, any or all of prior embodiments in this paragraph up through the second embodiment in this paragraph, wherein the temperature is between 700° C. and 900° C. An embodiment of the invention is one, any or all of prior embodiments in this paragraph up through the second embodiment in this paragraph, wherein the dehydrogenation catalyst particles are fresh catalyst when mixed with the additive particles. An embodiment of the invention is one, any or all of prior embodiments in this paragraph up through the second embodiment in this paragraph, wherein the dehydrogenation catalyst particles are deactivated catalyst. An embodiment of the invention is one, any or all of prior embodiments in this paragraph up through the second embodiment in this paragraph, wherein an oxygen concentration in the regenerator zone during treatment is at least 0.1 mol %.

A third embodiment of the invention is a mixture comprising dehydrogenation catalyst particles comprising gallium, and optionally platinum, on a support; and, additive particles comprising platinum on a support, wherein a level of platinum on each additive particle is at least 0.005 wt %; and when the dehydrogenation catalyst particles comprise platinum, wherein the level of platinum on the additive particle is higher than the platinum level on the dehydrogenation catalyst particles, wherein the mixture has an increased dehydrogenation activity after exposed to an oxygen containing environment at a temperature between 650° C. and 1,000° C., when tested in otherwise identical dehydrogenation reaction conditions. An embodiment of the invention is one, any or all of prior embodiments in this paragraph up through the third embodiment in this paragraph, wherein the support for the dehydrogenation catalyst particles and the support for the additive particles each, independently, comprise silica, alumina, silica-alumina, titania, zirconia, ceria, or amorphous aluminum phosphate. An embodiment of the invention is one, any or all of prior embodiments in this paragraph up through the third embodiment in this paragraph, wherein the mixture is fluidizable.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A process for activating a dehydrogenation catalyst, the process comprising:
providing dehydrogenation catalyst particles comprising gallium, and optionally platinum, on a support;
providing an additive particle comprising platinum, wherein a level of platinum on the additive particle is at least 0.005 wt %, and when the dehydrogenation catalyst particles comprise platinum, wherein the level of platinum on the additive particle is higher than a platinum level on the dehydrogenation catalyst particles;
treating the catalyst particles and the additive particle simultaneously in an oxygen containing gas environment at a temperature between 650° C. and 1,000° C.;
wherein an improvement of a dehydrogenation activity is observed in at least one of:
a) the dehydrogenation activity of the catalyst particles increases after the treatment and is higher than that of the catalyst particles before the treatment in otherwise identical dehydrogenation reaction conditions, or
b) the dehydrogenation activity of a mixture of the catalyst particles and the additive particle increases after the treating and is higher than that of the mixture before the treating in otherwise identical dehydrogenation reaction conditions.

2. The process of claim 1, wherein the treating temperature is between 700° C. and 900° C.

3. The process of claim 1, wherein the dehydrogenation catalyst particles are fresh catalyst when introduced to the additive particle.

4. The process of claim 1, wherein the dehydrogenation catalyst particles are deactivated catalyst.

5. The process of claim 1, wherein an oxygen concentration in the gas environment during treating is at least 0.1 mol %.

6. The process of claim 1, wherein the treating is performed in a catalyst regenerator.

7. The process of claim 1, wherein the treating is performed simultaneously as:
regenerating and re-heating the dehydrogenation catalyst particles.

8. The process of claim 1 further comprising:
separating the dehydrogenation catalyst particles from the additive particle after the treating.

9. The process of claim 1 wherein the support for the dehydrogenation catalyst particles comprises silica, alumina, silica-alumina, or amorphous aluminum phosphate.

10. The process of claim 1 wherein the additive particle further comprises a support, and the support for the additive particle comprises silica, alumina, silica-alumina, titania, zirconia, ceria, or amorphous aluminum phosphate.

11. The process of claim 1 wherein the mixture is fluidized during treating.

12. A process for activating a dehydrogenation catalyst, the process comprising:
providing dehydrogenation catalyst particles in a regeneration zone, wherein the catalyst particles comprise gallium, and optionally platinum, on a support, and wherein the regeneration zone is configured to receive an oxygen containing stream and an optional fuel stream and operates under conditions to re-heat the catalyst particles;
providing additive particle comprising platinum on a support, wherein a level of platinum on each additive particle is at least 0.005 wt %, and when the dehydrogenation catalyst particles comprise platinum, wherein the level of platinum on the additive particle is higher than a platinum level on the dehydrogenation catalyst particles,
treating the catalyst particles and additive particle simultaneously in the regeneration zone in an oxygen containing environment at a temperature between 650° C. and 1,000° C.;
wherein an improvement of a dehydrogenation activity is observed in at least one of:
a) the dehydrogenation activity of the catalyst particles increases after the treatment and is higher than that of the catalyst particles before the treating in otherwise identical dehydrogenation reaction conditions, or
b) the dehydrogenation activity of a mixture of the catalyst particles and the additive particle increases after the treating and is higher than that of the mixture before the treating in otherwise identical dehydrogenation reaction conditions.

13. The process of claim 12, wherein the treating is performed simultaneously as:
regenerating and re-heating the catalyst particles.

14. The process of claim 12, wherein the temperature is between 700° C. and 900° C.

15. The process of claim 12, wherein the dehydrogenation catalyst particles are fresh catalyst when mixed with the additive particles.

16. The process of claim 12, wherein the dehydrogenation catalyst particles are deactivated catalyst.

17. The process of claim 12, wherein an oxygen concentration in the regenerator zone during treatment is at least 0.1 mol %.

18. A mixture comprising:
dehydrogenation catalyst particles comprising gallium, and optionally platinum, on a support; and,
additive particles comprising platinum on a support, wherein a level of platinum on each additive particle is at least 0.005 wt %; and when the dehydrogenation catalyst particles comprise platinum, wherein the level of platinum on the additive particle is higher than a platinum level on the dehydrogenation catalyst particles,
wherein the mixture has an increased dehydrogenation activity after exposed to an oxygen containing environment at a temperature between 650° C. and 1,000° C., when tested in otherwise identical dehydrogenation reaction conditions.

19. The mixture of claim 18, wherein the support for the dehydrogenation catalyst particles and the support for the additive particles each, independently, comprise silica, alumina, silica-alumina, titania, zirconia, ceria, or amorphous aluminum phosphate.

20. The mixture of claim 18, wherein the mixture is fluidizable.
